(19) United States
(12) Patent Application Publication
(10) Pub. No.: US 2019/0231822 A1
(43) Pub. Date: Aug. 1, 2019

(54) METHODS OF REDUCING CHRONIC GRAFT-VERSUS-HOST DISEASE

(71) Applicant: Regents of the University of Minnesota, Minneapolis, MN (US)

(72) Inventors: Bruce R. Blazar, Golden Valley, MN (US); Jing Du, Minneapolis, MN (US); Robert Negrin, Palo Alto, CA (US)

(73) Assignee: Regents of the University of Minnesota, Minneapolis, MN (US)

(21) Appl. No.: 16/317,985

(22) PCT Filed: Jul. 14, 2017
(86) PCT No.: PCT/US2017/042217

§ 371 Date: Jan. 15, 2019

Related U.S. Application Data
(60) Provisional application No. 62/362,635, filed on Jul. 15, 2016.
(60) Provisional application No. 62/399,988, filed on Sep. 26, 2016.

(51) Int. Cl. A61K 35/17; A61K 9/00; A61P 37/06; A61K 31/7032

(52) U.S. Cl. CPC A61K 35/17; A61K 31/7032; A61P 37/06; A61K 9/0019

(57) ABSTRACT

Methods of reducing or reversing chronic graft-versus-host-disease (cGVHD) are provided herein.

### CROSS REFERENCE TO RELATED APPLICATIONS

This application claims the benefit of priority under 35 U.S.C. § 119(e) to U.S. Application No. 62/362,635, filed Jul. 15, 2016 and U.S. Application No. 62/399,988, filed Sep. 26, 2016.

### FEDERALLY SPONSORED RESEARCH OR DEVELOPMENT

This invention was made with government support under CA142106 awarded by the National Institutes of Health (NIH). The government has certain rights in the invention.

### TECHNICAL FIELD

This disclosure generally relates to methods of reducing or reversing chronic graft-versus-host disease (cGVHD).

### BACKGROUND

Chronic GvHD (cGVHD) can appear at any time following an allogenic transplant, including up to several years after the transplant. cGVHD can manifest itself in the skin, liver, eyes, mouth, lungs, gastrointestinal tract, neuromuscular system, or genitourinary tract. Patients who have undergone an allogeneic blood or bone marrow transplant have a greater risk for developing cGVHD, as are patients who have exhibited acute GVHD. Currently, cGVHD is treated with prednisone or other similar anti-inflammatory or immunosuppressive medications.

Methods to try to reduce or prevent cGVHD are being evaluated, including improvements in tissue typing, prophylactic immunosuppression of patients, and removal of donor T cells prior to transplant. The methods described herein provide a viable way to reduce or reverse cGVHD.

### SUMMARY

This disclosure provides for methods of reducing or reversing chronic graft-versus-host-disease (cGVHD).

In one aspect, methods of reducing chronic graft-versus-host-disease (cGVHD) in a patient are provided, where the patient is a recipient of a transplant from a donor. Such methods typically include identifying a patient suffering from cGVHD; providing donor iNKT cells; and administering the donor iNKT cells to the patient.

In some embodiments, the donor iNKT cells are administered to the patient one time. In some embodiments, the donor iNKT cells are administered to the patient two times. In some embodiments, the administering is by infusion (e.g., the donor iNKT cells can be administered to the patient by infusion). In some embodiments, the transplant is a bone marrow transplant, a hematopoietic stem cell transplant, or a progenitor cell transplant.

In some embodiments, such methods further include expanding the iNKT cells prior to the administering step. In some embodiments, such methods further include contacting the donor iNKT cells with RGI-2001 prior to the administering step.

In another aspect, methods of treating an autoimmune disease or an alloimmune disease in a patient are provided. Such methods typically include identifying a patient suffering from an autoimmune disease or an alloimmune disease; providing donor iNKT cells; and administering at least one dose of donor iNKT cells to the patient. Representative autoimmune diseases or alloimmune diseases include, without limitation, lupus, arthritic, immune complex glomerulonephritis, goodpasture, uveitis, multiple sclerosis and others.

In some embodiments, the donor iNKT cells are administered to the patient one time. In some embodiments, the donor iNKT cells are administered to the patient two times. In some embodiments, the administering is by infusion (e.g., the donor iNKT cells can be administered to the patient by infusion). In some embodiments, the transplant is a bone marrow transplant, a hematopoietic stem cell transplant, or a progenitor cell transplant.

In some embodiments, such methods further include expanding the donor iNKT cells prior to the administering step. In some embodiments, such methods further include contacting the iNKT cells with RGI-2001 prior to the administering step.

In still another aspect, a method of reducing chronic graft-versus-host-disease (cGVHD) in a patient is provided. Such a method typically includes administering a therapeutic amount of an agonist of iNKT cells to the patient.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 show the results of cell gating experiments, which demonstrates that the cell purity of iNKT cells after sorting was more than 95%.

FIG. 2 shows that isolated iNKT cells maintained cytokine-producing function based on an increase in IL4 (Panel A) and IFN-gamma (Panel B).

FIG. 3 shows a graph of the weight (Panel A) and survival (Panel B) of the mice.

FIG. 4 shows the results of the PFT experiments. Resistance (Panel A), elastance (Panel B) and compliance (Panel C) for mice were determined.

FIG. 5 shows the results of flow cytometry experiments to examine the types of cells present in the spleens of the animals treated with therapeutic iNKT. The amount of GC B cells (Panel A), Tfr cells (Panel B) and Tfh cells (Panel C) and the ratio of Tfh/Tfr cells (Panel D) were examined.

FIG. 6 show the results of cell gating experiments, which demonstrates that the cell purity of iNKT cells after sorting was more than 95%.

FIG. 7 shows that isolated iNKT cells maintained cytokine-producing function based on an increase of IL4 (Panel A) and IFN-gamma (Panel B) production.

FIG. 8 shows a graph of the weight (Panel A) and survival (Panel B) of the mice.

FIG. 9 shows that infusion with therapeutic iNKT cells improved cGVHD lung disease in a dose-dependent manner.

FIG. 10 shows that infusion with therapeutic iNKT cells reduced lung fibrosis in cGVHD mice.

FIG. 11 shows that infusion with therapeutic iNKT cells reduced collagen deposition in lung (top row) and liver (bottom row).

FIG. 12 shows TGF-beta staining of Tx4293 lung F4/80.

FIG. 13 shows the amounts of GC B cells (Panel A), Tfh cells (Panel B), follicular Treg cells (Panel C), total Treg cells (Panel D), and the ratio of Tfh/Tfr cells (Panel E) following infusion with iNKT cells.

FIG. 14 shows that infusion with iNKT cells decreased GC size (Panel B) and increased TFR density in GC (Panel D). In situ germinal center staining showed that iNKT infusion reduced germinal center area and also reduced Treg number per germinal center (Panel A).

FIG. 15 shows that infused iNKT cells can be identified in recipient tissues on day 59 (Panel A) in spleen, liver and lung (Panel B).

FIG. 16 show the results of cell gating experiments, which demonstrates that the cell purity of iNKTcells after sorting was more than 95%.

FIG. 17 are graphs showing the weight (Panel A) and survival (Panel B) of the mice.

FIG. 18 shows the analysis of lung disease using pulmonary function.

FIG. 19 show the results of flow cytometry analysis.

FIG. 20 show staining of the CD1d-Tetramer (Panel A), a graph of the iNKT population (Panel B), and staining of H2b (Panel C) in various mice (e.g., B10BR mice, B6 mice, BM only mice, and cGVHD mice). Similarly, flow analysis for IL-4 (Panels D and E) and IFN-gamma (Panels F and G) was performed in the various mice.

FIG. 21 is a schematic showing an experimental plan for determining which Treg compartment is required to reduce cGVHD.

FIG. 22 are graphs and images showing that infused iNKT cells disseminated in the recipients and were identified from recipients' lung, liver and spleen.

FIG. 23 are graphs showing that iNKT cells controlled the spontaneous germinal center reactions that drive cGVHD pathogenesis by expanding donor Tregs and increasing follicular Treg density in the germinal center areas.

FIG. 24 shows data that demonstrates that therapeutic iNKT cell infusion reversed established cGVHD. B10.BR mice were conditioned by 2 doses of cyclophosphamide (120 mg/kg body weight, ip) on day −3 and day −2 of transplantation. On day −1, B10.BR mice were irradiated (830 Gy by x-ray) then infused with T-cell depleted (TCD) BM only or with 75,000 purified T cells to induce cGVHD. Panels 24A and 24B show that, on day 28 after transplantation, splenocytes were harvested from BM-only mice and cGVHD mice and naïve mice of donor and recipient strains. Cells were stained with fluorochrome conjugated PBS57-CD1D tetramer, anti-CD4, anti-TCR-beta and viability dye. iNKT cells were identified by CD4+ TCR-beta+ PBS57-CD1D+ live cells. Panel 24A shows the gating of iNKT cells. Cells were gated on live CD4+ T cells. Panel 24B shows that the frequency of iNKT is significantly reduced in cGVHD mice. Panels 24C and 24D show that, on day 28 and day 42 post-transplantation, FACS-sorted CD45.1 B6 iNKTs were infused to some cGVHD mice at a lower (50K) or higher (100K) dose. Panel 24C shows that pulmonary function tests (PFTs), including resistance, elastance and compliance, were performed on day 56 post transplantation. iNKT infusion significantly improved the pulmonary function. Panel 24D shows that hydroxyproline was measured in the lungs of mice from FIG. 24C. iNKT infusion at the 100K cell dose significantly reduced hydroxyproline. Panel 24E shows that trichrome staining, which identifies collagen, was performed on cryosections of lungs and imaged at 200×. Panel 24F shows that collagen deposition was quantified by measuring the blue area by Fiji software. iNTK infusion significantly reduced collagen deposition in the lung. Panel 24G shows that cGVHD was established as previously described. Mice received Balb/c iNKT cells on days 28 and 42. Balb/c iNKT cells reverse cGVHD. Panel 24H shows cryo-sections of spleen (day 56) were stained with fluorochrome conjugated anti-CD4 (FITC), anti-Foxp3 (eFluor660) and peanut-agglutinin (PNA) (Rhodamine) and imaged by Olympus FV1000 system at 400×. Dotted lines delineate GC areas by PNA staining. Panel 241 shows that follicular Tregs were identified as CD4+ Foxp3+ cells within the GC areas. Panel 24J shows that average GC size was decreased by iNKT. Panel 24K shows that follicular Treg density was increased by iNKT. Panel 24L-24M shows that splenic GC B cells and follicular Tregs frequencies were determined by flow cytometry on day 55 post-transplantation. iNKT infusion decreased GC B and increased follicular Treg frequencies. Unpaired student t-test was used when comparing the two groups. Data shown are representative of 2-4 independent experiments with 5-8 mice per group, except Panel 24G, which represents 1 experiment with 5-8 mice. Significance: *P<0.05; **P<0.01; ***P<0.001; ****P<0.0001.

FIG. 25 shows data that demonstrates that iNKT reversed cGVHD through donor Treg expansion and prevented the onset of cGVHD. Panels 25A-25C show that cGVHD was established as per FIG. 24, except that BM and T cells were harvested from B6.Foxp3.Luci.DTR-4 mice. Group 1 and 2 are BM only and cGVHD control, respectively, as described previously. Groups 3-5 are cGVHD mice that received diphtheria toxin (DT) (Group 3), iNKT infusion on day 28 and day 42 (Group 4) or iNKT infusion and DT (Group 5). Panel 25A shows that, on day 43, mice were imaged by a Spectrum In Vivo Imaging system. Panel 25B shows that quantification of the BLI signal indicated depletion of Tregs by DT and expansion of Tregs by iNKT infusion. Panel 25C shows that PFTs were assessed as described in FIG. 24. Treg depletion by DT injection completely abolished iNKTs efficacy. Panel 25D shows that iNKT cells (white arrow) were identified by CD45.1+ in GC area. Panel 25E shows mice that were transplanted as per FIG. 24. iNKT cells from wildtype, CXCRS−/− or IL4−/− mice were infused to transplanted mice on day 28 and 42. iNKT cells from CXCRS−/− or IL4−/− mice lost the ability to reverse cGVHD. Panel 25F shows that cGVHD mice were infused with B6 iNKTs either on day 1 and day 14 (prophylaxis) or on day 28 and day 42 (therapy). Prophylatic iNKT infusion completely blocked cGVHD. Panel 25G shows that prophylatic or therapeutic RGI2001 (2.5 microgram/mouse) was given to transplanted mice. PFTs suggest RGI2001 prevented and reversed cGVHD. 5-8 mice per group were analyzed for each assay. Significance: *P<0.05; **P<0.01; ***P<0.001; ****P<0.0001.

### DETAILED DESCRIPTION

This disclosure describes the administration of invariant natural killer T cells (iNKT cells) as a therapeutic intervention for chronic graft versus host disease (cGVHD). As described herein, administration of iNTK cells disrupts the pathogenic immune response in cGVHD and may act in a similar manner in diseases mediated by autoimmune or alloimmune responses such as lupus, arthritic, immune complex glomerulonephritis, goodpasture, uveitis, multiple sclerosis and others.

Chronic GVHD (cGVHD) is different from acute GVHD (aGVHD), and is defined by clinical markers and is not dependent on the timing of the transplantation. In fact, cGVHD and aGVHD can occur simultaneously. The criteria for diagnosing and scoring the severity of cGVHD is described in Jagasia et al. (2015, Biol. Blood Marrow Transplant., 21(3):389-401), which breaks the clinical markers down by organ.

Current strategies for treating cGVHD and other autoimmune or alloimmune diseases include infusion of Treg cells, injection of antibodies, and/or chemotherapies. The methods described in this disclosure allow for the use of a small amount of iNKT cells as compared to current methods that require the use of a large amount of Treg cells. The methods described in this disclosure also require fewer treatment doses and have much less toxicity than injection of antibodies and different forms of chemotherapies.

Based upon an increase in IL-4 and IL-10 production in the presence of iNKT cells, iNKT cells would be expected to worsen cGVHD. Surprisingly, however, infusion with iNKT cells demonstrated IL-4-dependent protection against the disease. This is the first description of the use of iNKT cells to treat cGVHD.

The methods described herein can be used to reduce chronic graft versus host disease (cGVHD) in a patient. In some instances, the patient has received a bone marrow transplant, a hematopoietic stem cell transplant, or a progenitor cell transplant, from a donor. In some instances, the patient has received a solid organ transplant (e.g., kidney, liver, heart, lung, etc.) from a donor. In addition, the methods described herein can be used to treat an autoimmune or alloimmune disease (e.g., chronic alloimmune or autoimmune responses). It would be appreciated that, for autoimmune diseases, iNKT cells can be obtained from the patient and expanded ex vivo or iNKT cells can be obtained from an appropriate donor including a cadaveric donor. It would be appreciated that the iNKT cells do not need to be from the original donor but can instead come from a third party donor. Representative autoimmune and alloimmune diseases include, without limitation, lupus, arthritic, immune complex glomerulonephritis, goodpasture, uveitis, multiple sclerosis and others.

iNKT cells can be obtained using known methods. The markers typically associated with human iNKT cells include, without limitation, V(alpha)24-J(alpha)18 and V(beta)11. It would be understood that one or more antibodies (e.g., one or more labeled antibodies) can be used to obtain iNKT cells. See, for example, Montoya et al. (2007, Immunology, 122(1):1-14), which describes a monoclonal antibody that is specific for the invariant CDR3 loop of the human canonical V(alpha)24-J(alpha)18 TCR alpha chain, referred to as 6B11. See, for example, Leveson-Gower et al., 2011, Blood, 117:3220-9; Hongo et al., 2012, Blood, 119:1581-9; and Cameron et al., 2015, J. Immunol., 195:4604-14. Representative methods of obtaining iNKT cells from mice also are described herein.

As described herein, the iNKT cells can be administered once or more than once (e.g., twice, three times, four times, or more) to a patient. The iNKT cells can be administered in an amount ranging from about 0.1×10⁶ /kg of patient weight up to about 40×10⁶/kg of patient weight (e.g., about 0.1×10⁶ /kg to about 20×10⁶ /kg; about 1×10⁶ /kg to about 10×10⁶ /kg; about 5×10⁶ /kg to about 40×10⁶ /kg; about 10×10⁶ to about 25×10⁶ /kg; about 15×10⁶ to about 30×10⁶ /kg). There are no known dose limiting effects of iNKT cells, but the methods described herein require an amount of iNKT cells that is much less than similar therapeutic methods that utilize Treg cells instead.

The iNKT cells can be administered at any time before or after transplant. For example, iNKT cells can be administered prophylactically to a patient prior to receiving a transplant (e.g., minutes, hours or days before the transplant). Additionally or alternatively, iNKT cells can be administered therapeutically to a patient after receiving a transplant. For example, iNKT cells can be administered with a transplant, or within minutes or hours of receiving a transplant, or even within weeks or months or years of receiving a transplant. In most instances, it is desirable to introduce the iNKT cells into the recipient as soon as they are harvested from the donor or shortly thereafter (e.g., within 2, 6, 8, 12, or 24 hours of harvesting such cells).

Typically, the iNKT cells are administered to the patient by infusion. Other methods can be used to administer iNKT cells to a patient, however, including, without limitation, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

It would be appreciated that the iNKT cells can be expanded. Methods of expanding iNKT cells are known in the art. Typically, expansion would occur after the iNKT cells are obtained from the donor but before the iNKT cells are administered to the patient (i.e., ex vivo), but expansion could occur in vivo, for example, by administering one or more cytokines (e.g., IL-2, IL-17, IL-15). A representative compound that can cause expansion of iNKT cells is the small molecule RGI-2001 (see, for example, Duramad et al., 2011, Biol. Blood Marrow Transplant., 17(8):1154-68). As used herein, expansion of iNKT cells typically refers to an increase in number, but expansion of iNKT cells also can refer to an increase in activity or potency of the cells.

In addition to the methods described herein, methods also are provided for increasing the frequency of endogenous iNKT cells in vivo or increasing or sustaining the frequency of infused or endogenous iNKT cells in vivo. Such methods can utilize compounds such as, without limitation, cytokines (e.g., IL-2, IL-17, IL-15) or alpha-galactosyl ceramide. Similarly, agonists of iNKT cells can be administered to a subject. Agonists of iNKT cells are known in the art and include, without limitation, synthetic agonists (see, e.g., Cerundolo et al., 2010, Curr. Opin. Immunol., 22(3):417-24), alpha-galactosylceramides and beta-mannosylceramides (see, e.g., O'Konek et al., 2011, J. Clin. Invest., 121(2):683-94; Aspeslagh et al., 2011, The EMBO J., 30(11):2294-305), and threitolceramide (see, e.g., Silk et al., 2008, J. Immunol., 180(10):6452-6). See, also, Cerundolo et al., 2009, Nature Rev., 9(1):28-38.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Experimental Plan for Tx4257

iNKT Cell Isolation Procedure:

Splenocytes were harvested from B6 donor mice and CD19+, CD220+, and CD8+ cells were depleted using MACS cell separation technology. The remaining cells were stained with antibodies to the following markers: CD4-V500, TCR-b-Percp-Cy5.5, FVD-af780, and CD1d Tetramer-PE. MACS positive selection was performed on PE-positive cells, and the cells were sorted on CD1d-PBS-57 tetramer+ CD4+ TCR-beta+ live cells. The isolated iNKT cells were infused into mice or functionally analyzed for cytokine production. For functional analysis, cells were stimulated for 4 hours in a CO2 incubator with cell stimulation cocktail (eBioscience) and flow cytometry was used to evaluate cytokine production.

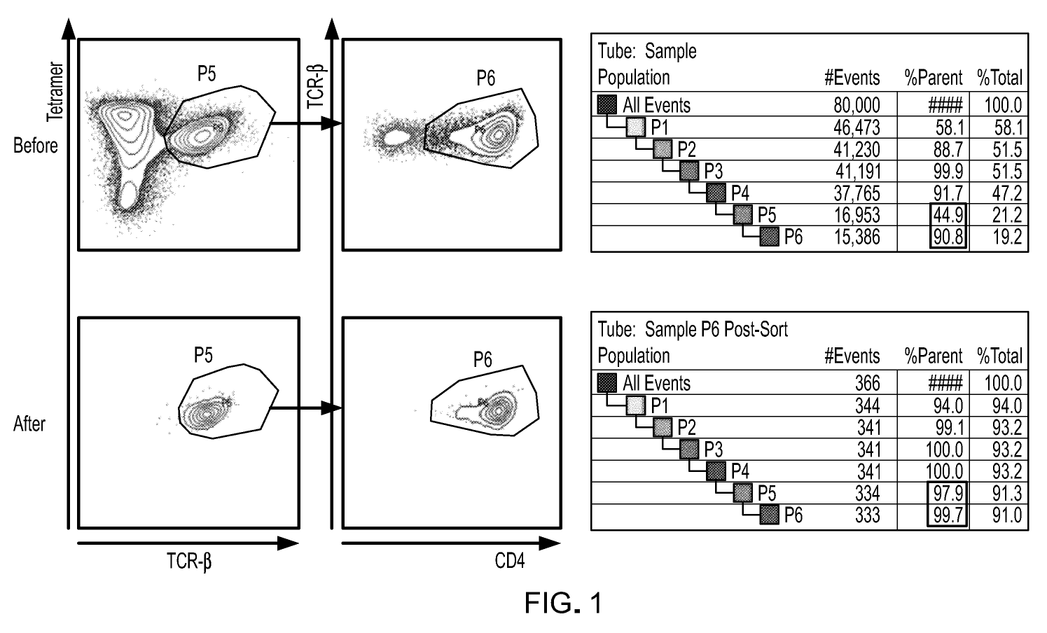

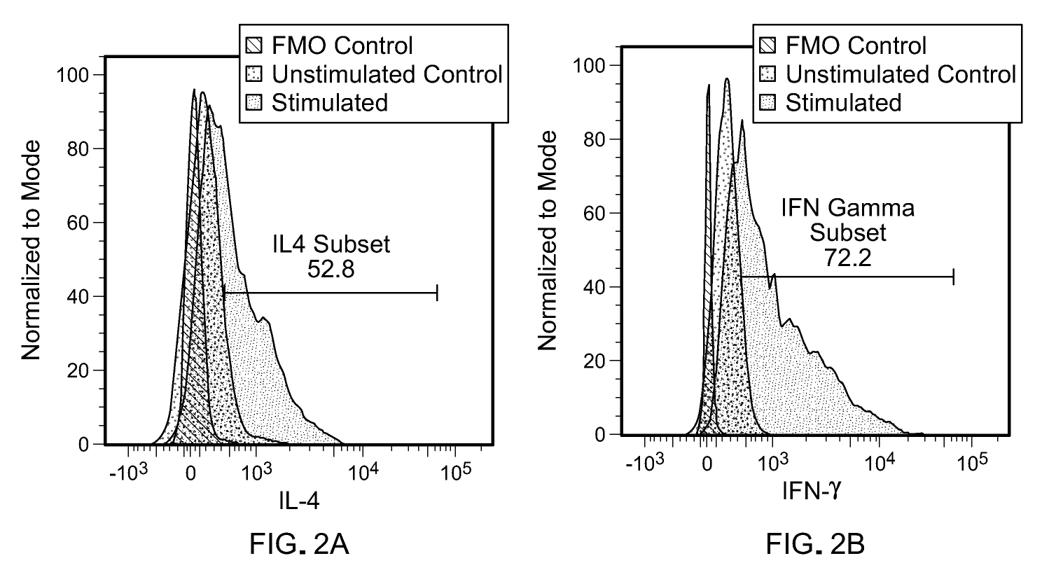

FIG. 1 demonstrates that the cell purity of iNKT cells after sorting was more than 95%, and FIG. 2 shows that isolated iNKT cells maintained cytokine-producing function. As shown in FIG. 2, after stimulation, there was a significant increase of IL4 (Panel A) and IFN-gamma (Panel B) production.

### In Vivo Experiments:

Four groups of mice (n=10) were treated as described below in Table 1. Briefly, B10.BR mice were irradiated on day 0, and transplanted on day 1 with bone marrow from B6 mice. iNKT cells were infused at day 28 and day 42. On day 56, weight and survival for each recipient was noted, and pulmonary function tests (PFT) of 5-7 mice from each group were performed. A hydroxyproline assay also was performed on day 56, as were trichrome staining of lung, liver, spleen, thymus and colon. In addition, splenocytes were harvested and stained for markers of GC B cells, T follicular helper cells (Tfh) and T follicular regulatory cells (Tfr), and gated on live B cells and live T cells. In addition, cGVHD pathogenesis depends on increased germinal center (GC) reaction (e.g., increased GC B cells and follicular helper T cells and decreased follicular regulatory T cells). Therefore, these cell populations were examined as biomarkers of cGVHD pathogenesis.

TABLE 1

| | | | | | | BM | T cell | |
|---|---|---|---|---|---|---|---|---|
| | | | | Cy | X-ray | (TCD) | dose | |
| Group | N | Donor | Recipient | (day −3, −2) | (day 0) | (day 0) | (day 0) | Treatment |
| 1 | 10 | B6 WT | B10.BR | 120 mg/kg | 830 | 10⁷ | — | — |
| 2 | 10 | B6 WT | B10.BR | 120 mg/kg | 830 | 10⁷ | 70000 | — |
| 3 | 10 | B6 WT | B10.BR | 120 mg/kg | 830 | 10⁷ | 70000 | 50K iNKT cells on |
| | | | | | | | | day 28 and day 42 |
| 4 | 10 | B6 WT | B10.BR | 120 mg/kg | 830 | 10⁷ | 70000 | 50K iNKT cells on |
| | | | | | | | | day 28 |

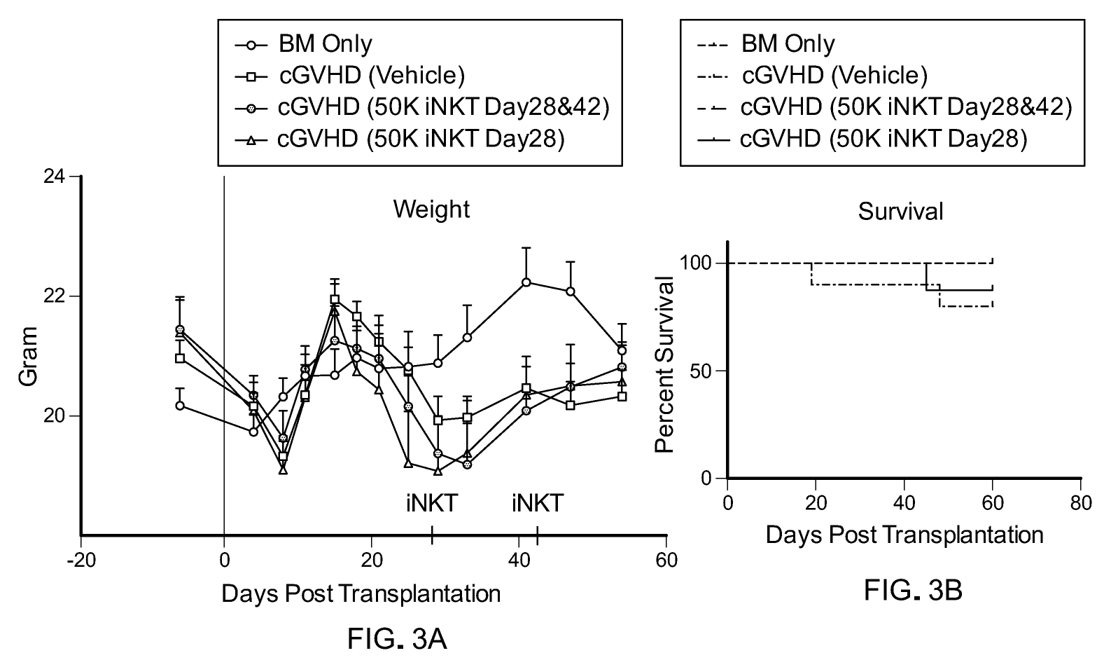

FIG. 3 shows a graph of the weight (Panel A) and survival (Panel B) of the mice. As can be seen by FIG. 3, the results of these experiments suggest that chronic GVHD, rather than acute GVHD, is more prevalent in mice, where more weight loss and death would occur.

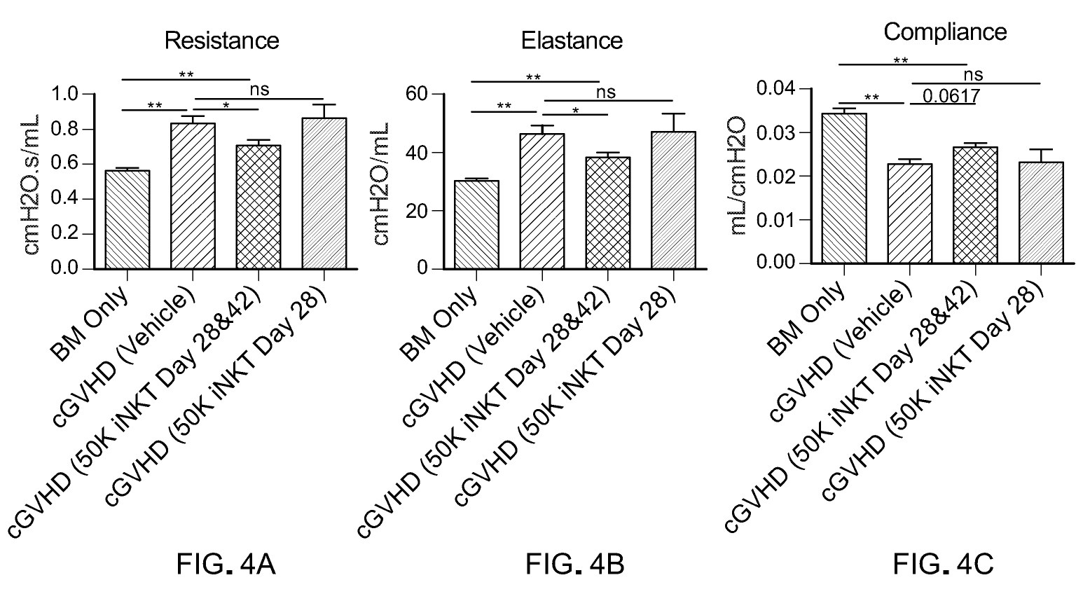

FIG. 4 shows the results of the PFT experiments. Resistance (Panel A), elastance (Panel B) and compliance (Panel C) were determined. The results shown in FIG. 4 demonstrate that two infusions with therapeutic iNKT improved pulmonary function. 50K iNKT infusion on day 28 and day 42 increased pulmonary function significantly, but didn't completely restore lung function, while a single dose of 50K iNKT on day 28 didn't significantly improve cGVHD.

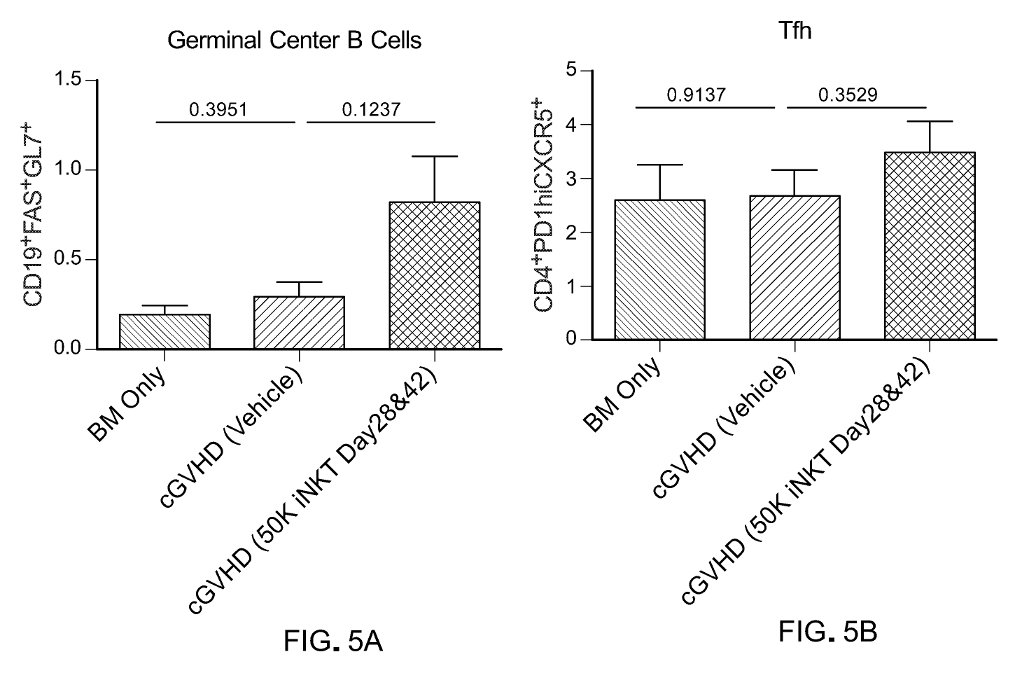

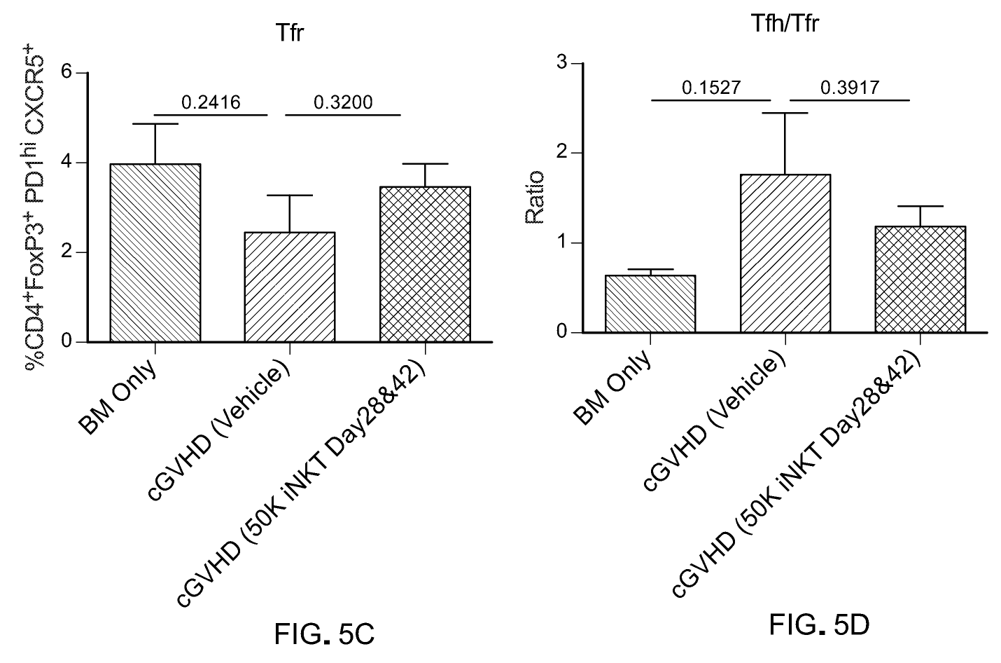

FIG. 5 shows the results of flow cytometry experiments to examine the types of cells present in the spleens of the animals treated with therapeutic iNKT. The results shown in FIG. 5 demonstrate that infusion with iNKT cells decreased the ratio of Tfh/Tfr cells (Panel D). Increased GC B cells and Tfh cells and decreased Tfr cells are expected during cGVHD, however, this was not observed in this experiment (Panels A, B and C, respectively). There was a slight trend of an increase of Tfr cells (Panel B) and a decrease of the Tfh/Tfr ratio in the treatment group (Panel D), but it was not significant.

These experiments demonstrated that flow-sorted iNKT cells have high purity and maintain their cytokine producing function, and that 50k donor iNKT cells infusion on day 28 and 42, but not on day28 only, significantly improved cGVHD. The role of iNKT infusion on germinal center reaction and follicular Treg were not entirely clear from these flow result.

### Example 2

### Experimental Plan for Tx4293

iNKT Cell Isolation Procedure:

Splenocytes were harvested from CD45.1 B6 donor mice and CD19+, CD220+, and CD8+ cells were depleted using flow cytometry. The remaining cells were stained with antibodies to the following markers: CD4-V500, TCR-b-Percp-Cy5.5, FVD-af780, and CD1d Tetramer-PE. MACS positive selection was performed on PE-positive cells, and the cells were sorted on CD1d-PBS-57 tetramer+ CD4+ TCR-beta+ live cells. The isolated iNKT cells were infused into mice or stimulated with PMA for 4 hours followed by flow cytometry to analyze cytokine production.

For stimulation of iNTK cells, cells isolated as described herein were seeded into two wells of a 24-well plate in 1 ml of complete cell culture media. 2 μl of cell stimulation cocktail plus cytokine transport inhibitor (eBioscience) was added to one of the wells. Cells were incubated in a CO₂ incubator for 4 hrs, and then harvested for cytokine production.

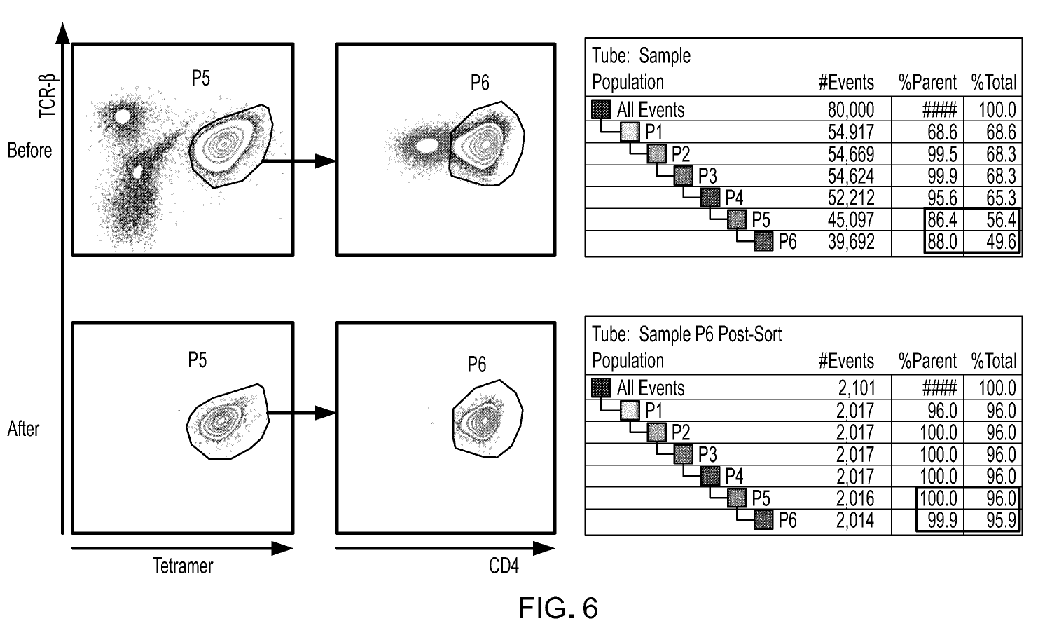

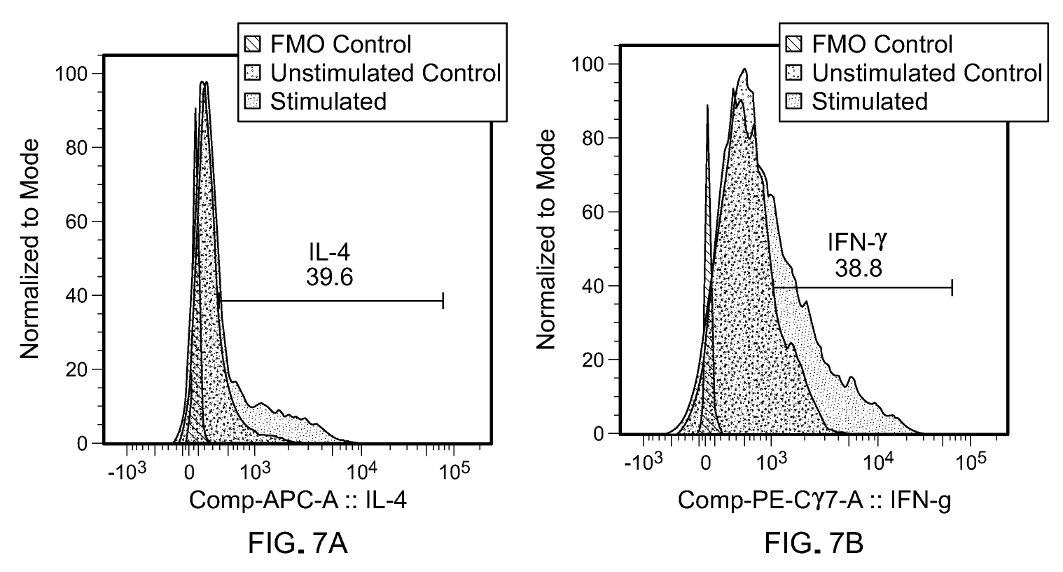

FIG. 6 demonstrates that the cell purity of iNKT cells after sorting was more than 95%, and FIG. 7 shows that isolated iNKT cells maintained cytokine-producing function. As shown in FIG. 7, after stimulation, there was a significant increase of IL4 (Panel A) and IFN-gamma (Panel B) production by the sorted iNKT cells. It was noted that cytokine production in the un-stimulated control (blue) was slightly higher than with Tx4257, which could be the result of tetramer binding-induced iNKT activation or the result of a longer incubation time before being analyzed by the flow cytometry.

### In Vivo Experiments:

Four groups of mice (n=10) were treated as described below in Table 2. Briefly, B10.BR mice were irradiated on day 0, and transplanted on day 1 with bone marrow from B6 WT mice. iNKT cells were infused at day 28 and day 42. On day 56, weight and survival for each recipient was noted, and pulmonary function tests (PFT) of 5-7 mice from each group were performed on day 60. Trichrome staining of lung and liver was performed. Lungs from 5-7 mice from each group were measured for hydroxyproline content, and frozen tissue sections from 4-6 mice were stained for collagen, and the collagen areas were quantified by image J. In addition, splenocytes from 5-7 mice from each group were harvested and stained for markers of GC B cells, T follicular helper cells (Tfh) and T follicular regulatory cells (Tfr), and gated on live B cells and live T cells. Therefore, these cell populations were examined as biomarkers of cGVHD pathogenesis.

TABLE 2

| | | | | | | BM | T cell | |
|---|---|---|---|---|---|---|---|---|
| | | | | Cy | X-ray | (TCD) | dose | Treatment |
| Group | N | Donor | Recipient | (d −3, −2) | (d −1) | (day 0) | (day 0) | (day 28 and 42) |
| 1 | 10 | B6 WT | B10.BR | 120 mg/kg | 830 | 10⁷ | — | |
| 2 | 10 | B6 WT | B10.BR | 120 mg/kg | 830 | 10⁷ | 72500 | — |
| 3 | 10 | B6 WT | B10.BR | 120 mg/kg | 830 | 10⁷ | 72500 | 50K iNKT |
| 4 | 10 | B6 WT | B10.BR | 120 mg/kg | 830 | 10⁷ | 72500 | 100K iNKT |

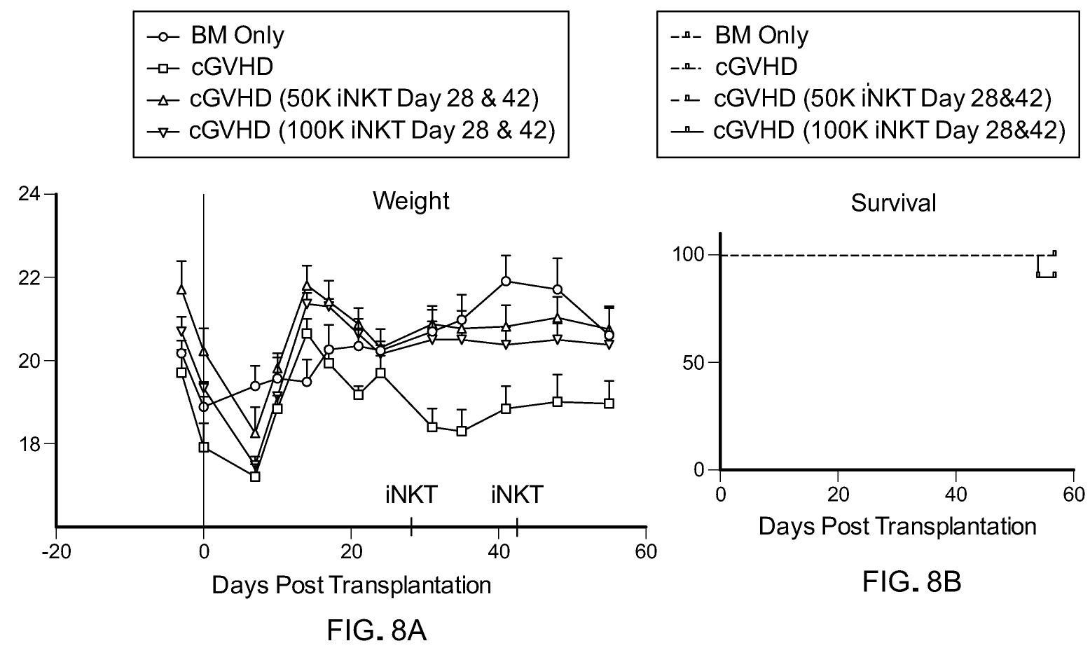

FIG. 8 shows a graph of the weight (Panel A) and survival (Panel B) of the mice. As can be seen by FIG. 8, the results of these experiments suggest that chronic GVHD, rather than acute GVHD, is more prevalent in mice, where more weight loss and death would occur.

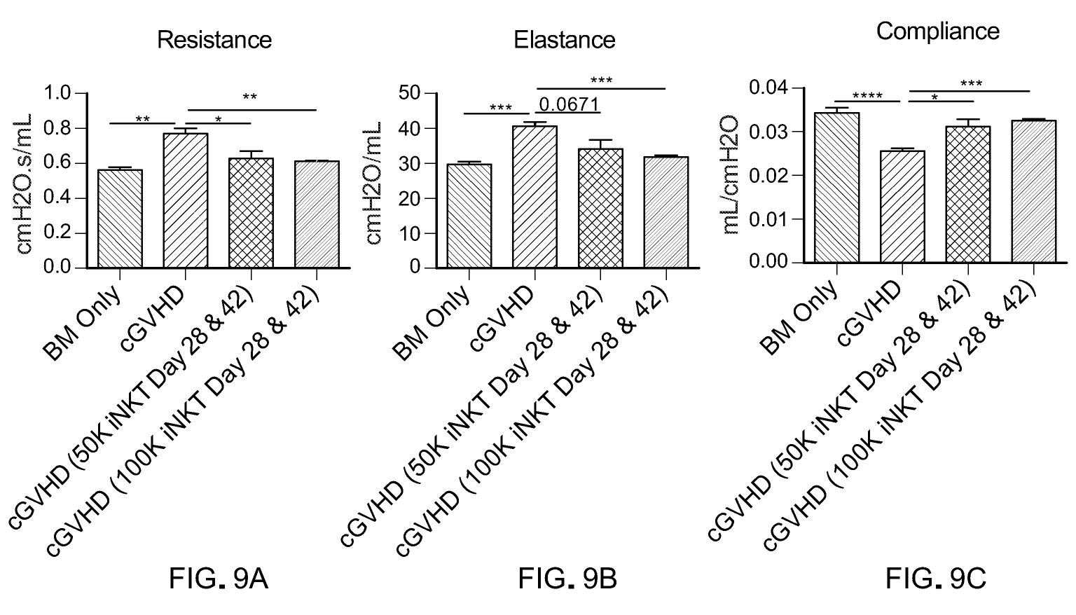

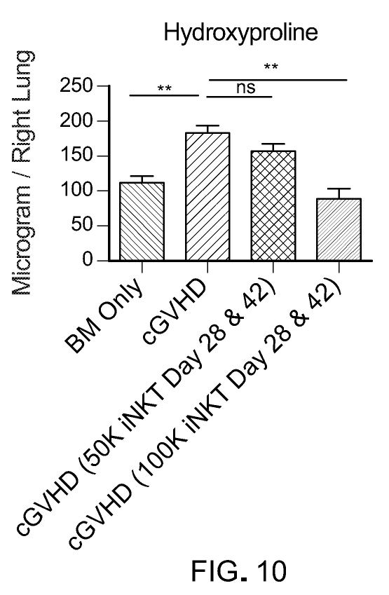

FIG. 9 shows that infusion with therapeutic iNKT cells improved cGVHD lung disease in a dose-dependent manner, and FIG. 10 shows that infusion with therapeutic iNKT cells reduced lung fibrosis in cGVHD mice.

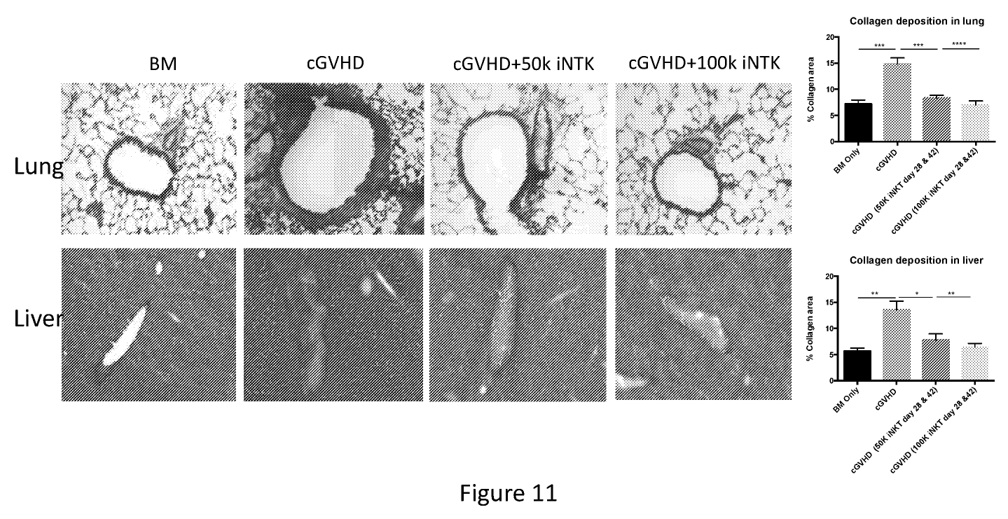

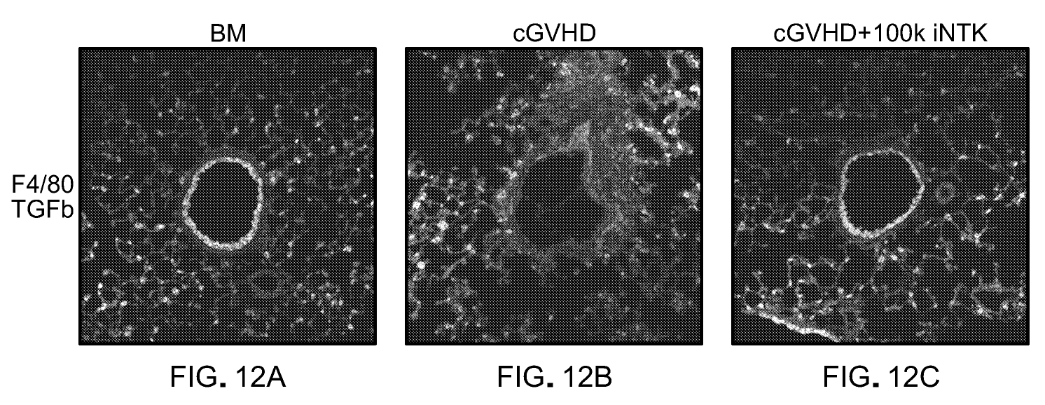

FIG. 11 shows that infusion with therapeutic iNKT cells reduced collagen deposition in lung (top row) and liver (bottom row), and FIG. 12 shows TGF-beta staining of Tx4293 lung F4/80. It was previously determined that cGVHD is associated with increased macrophage infiltration and TGF-beta deposition. FIG. 12 shows that infusion with iNKT cells reduced macrophage and TGF-beta in the peri-bronchial area (although F4/80 also stained bronchial epithelial cells, even with FC blocking).

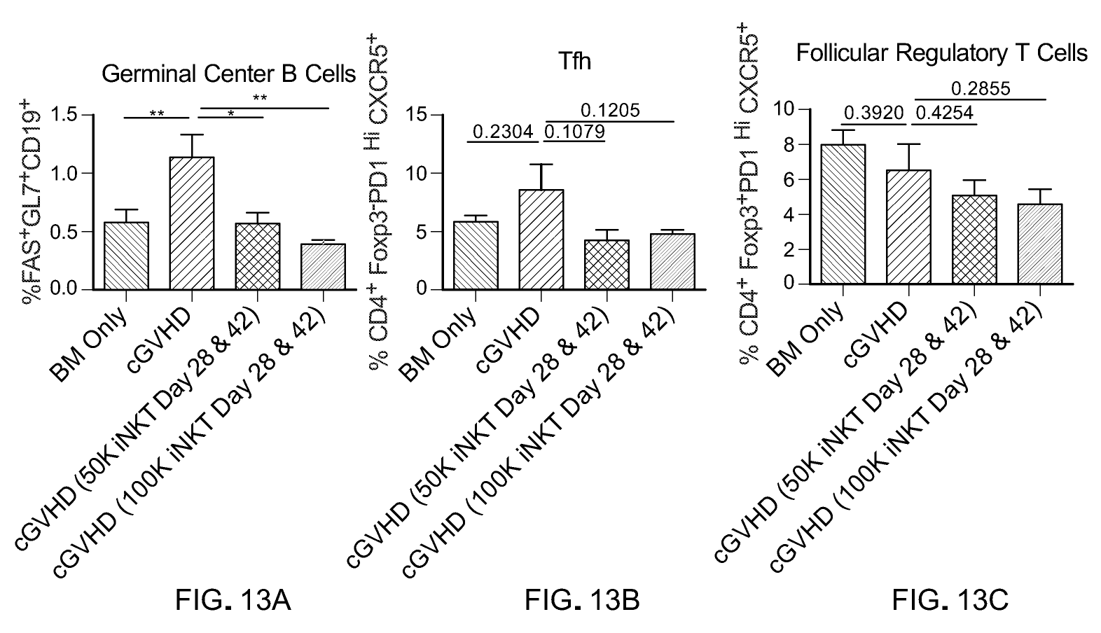

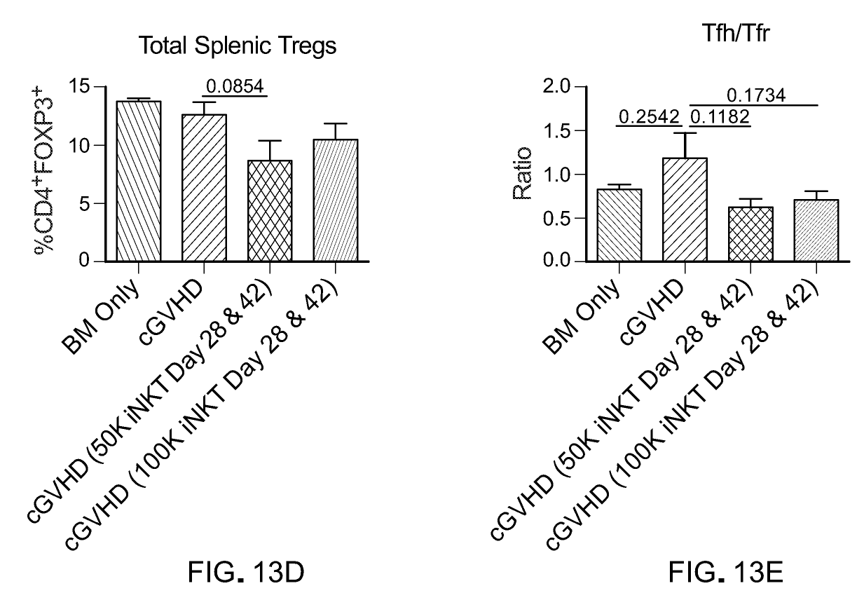

FIG. 13 shows that infusion with iNKT cells slightly reduced the ratio of Tfh/Tfr cells (Panel E). Flow analysis of day 60 splenocytes showed that iNKT infusion reduced germinal center reaction (GC B cells (Panel A) and Tfh cells (Panel B)). No significant change on total Treg cells (Panel D) or follicular Treg (Panel C) was observed.

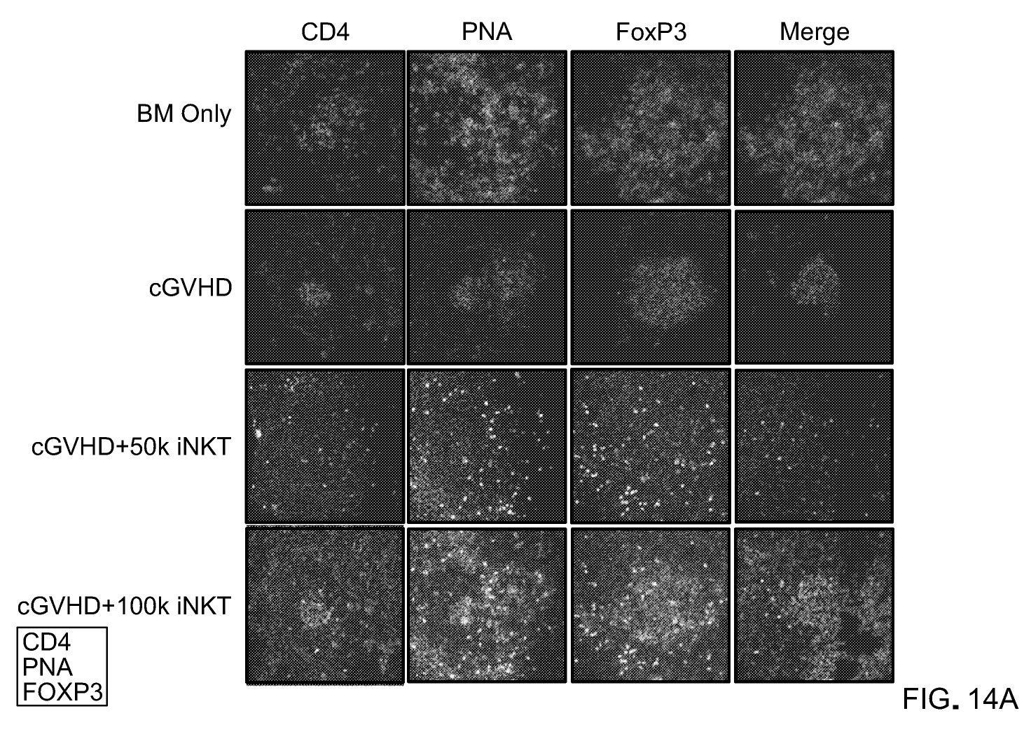

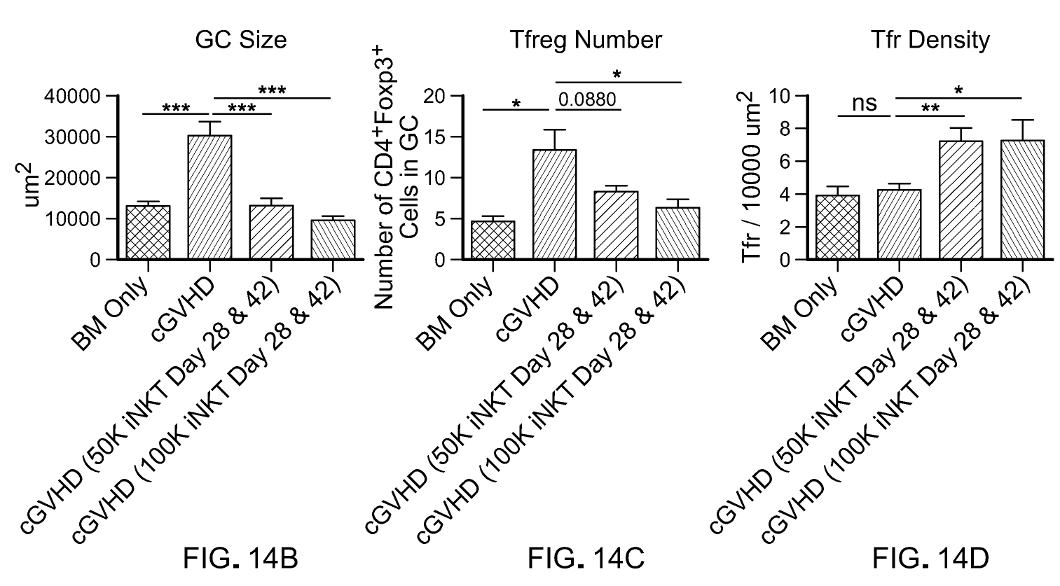

FIG. 14 shows that infusion with iNKT cells decreased GC size (Panel B) and increased TFR density in GC (Panel D). In situ germinal center staining showed that iNKT infusion reduced germinal center area and also reduced Treg number per germinal center (Panel A).

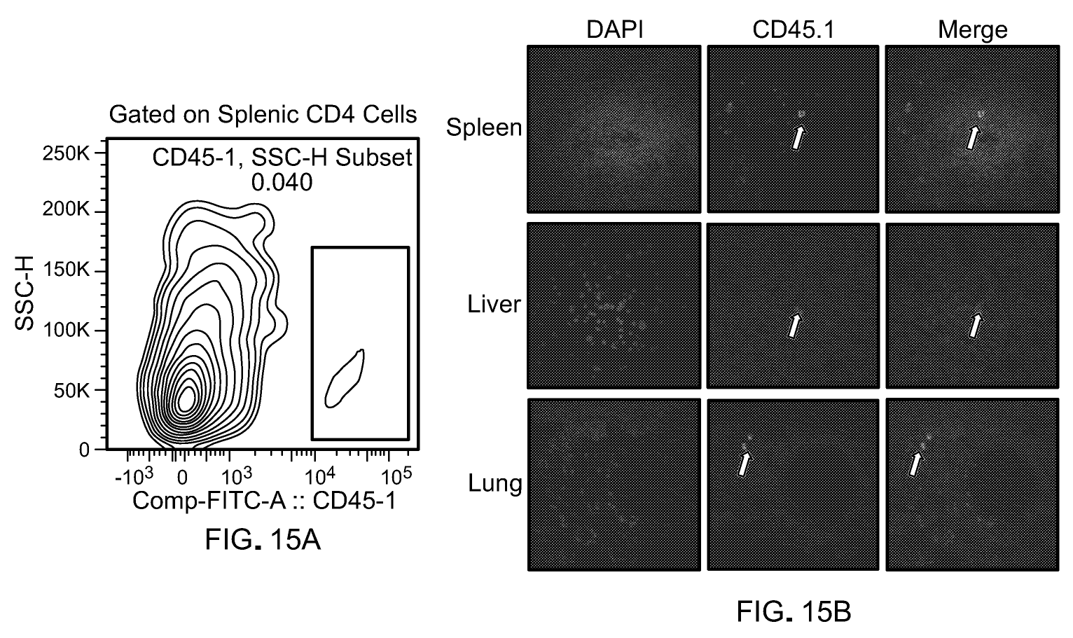

FIG. 15 shows that infused iNKT cells can be identified in recipient tissues on day 59 (Panel A). In this experiment, donor CD45.1⁺ iNKT cells were detected in spleen, liver and lung (Panel B).

In summary, these experiments demonstrated that iNKT cells reduced cGVHD lung disease in a dose-dependent manner, and that the effect of iNKT on cGVHD is associated with decreased germinal center reaction (e.g., decreased GCB, Tfh and Tfh/Tfr ratio, and increased Tfr density). These experiments also demonstrated that infused iNKT T cells can be identified in spleen, liver and lung. Lung, liver, spleen and colon samples from day 59 were obtained and frozen.

### Example 3

### Experimental Plan for Tx4352

Pharmacologic expansion of donor-derived, naturally occurring CD4(+)Foxp3(+) regulatory T cells reduces acute graft-versus-host disease lethality without abrogating the graft-versus-leukemia effect in murine models (see Duramad et al., 2011, Biol. Blood Marrow Transplant, 17(8):1154-68). Therefore, it was hypothesized that pharmacologic activation of iNKT cells using RGI-2001 would improve murine cGVHD. RGI-2001 is a liposomal formulation of a-galactosylceramide that can be presented to iNKT cells with a CD1d molecule.

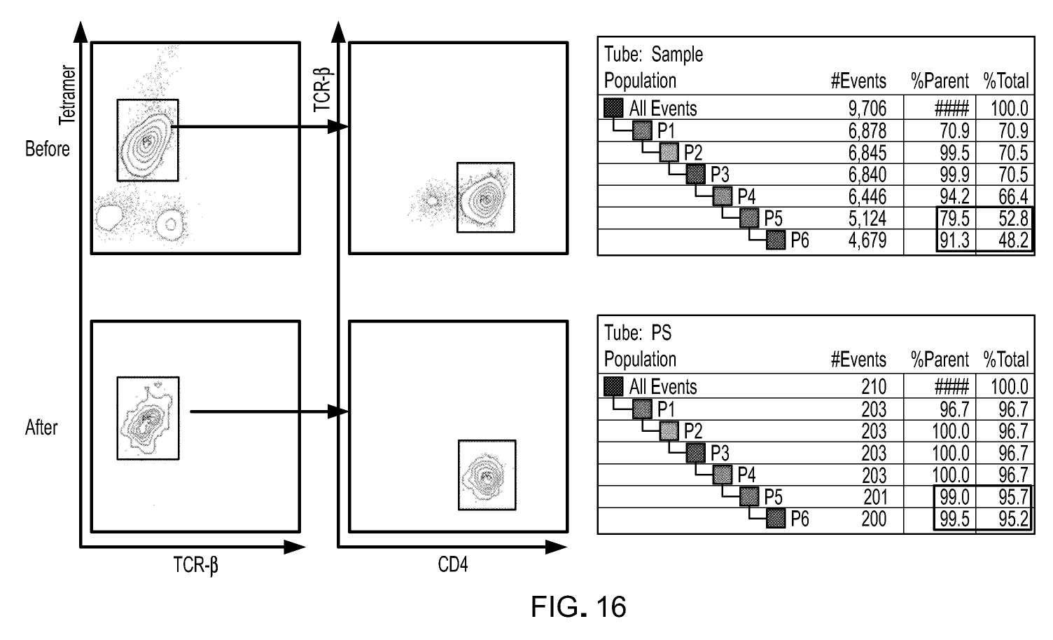

iNKT cells were isolated as described herein, and FIG. 16 demonstrates that the cell purity of iNKTcells after sorting was more than 95%. The treatment regimen is shown below in Table 3. For treatment with RGI-2001, 100 mcg/kg of the RGI-2001 or of the control vehicle was intravenously injected at the indicated time points.

TABLE 3

| | | | | Cy | | BM | T cell | |
|---|---|---|---|---|---|---|---|---|
| Group | N | Donor | Recipient | (d −3, −2) | X-Ray | (TCD) | Dose | Treatment |
| 1 | 8 | CD45.1 B6 | B10.BR | 120 mg/kg | 830 | 10⁷ | — | — |
| 2 | 12 | CD45.1 B6 | B10.BR | 120 mg/kg | 830 | 10⁷ | 72500 | — |
| 3 | 10 | CD45.1 B6 | B10.BR | 120 mg/kg | 830 | 10⁷ | 72500 | 100K CD45.1 iNKT |
| | | | | | | | | (day 1 & 14) |
| 4 | 10 | CD45.1 B6 | B10.BR | 120 mg/kg | 830 | 10⁷ | 72500 | 100K CD45.1 iNKT |
| | | | | | | | | (day 28 & 42) |
| 5 | 10 | CD45.1 B6 | B10.BR | 120 mg/kg | 830 | 10⁷ | 72500 | RGI2001 |
| | | | | | | | | (day 1 & 14) |
| 6 | 10 | CD45.1 B6 | B10.BR | 120 mg/kg | 830 | 10⁷ | 72500 | RGI2001 |
| | | | | | | | | (day 28 & 42) |

On day 56, weight and survival for each recipient was noted, and pulmonary function tests (PFT) of 5-7 mice from each group were performed. A hydroxyproline assay also was performed on day 56, as were trichrome staining of lung, liver, spleen, thymus and colon. In addition, splenocytes were harvested on day 56 and stained for markers of Tregs, Tfh, Tfr, GC B cells, donor MDSCs, iNKT cells, and gated on live B cells and live T cells. IL-4 production also was evaluated in liver and spleen. Therefore, these cell populations were examined as biomarkers of cGVHD pathogenesis.

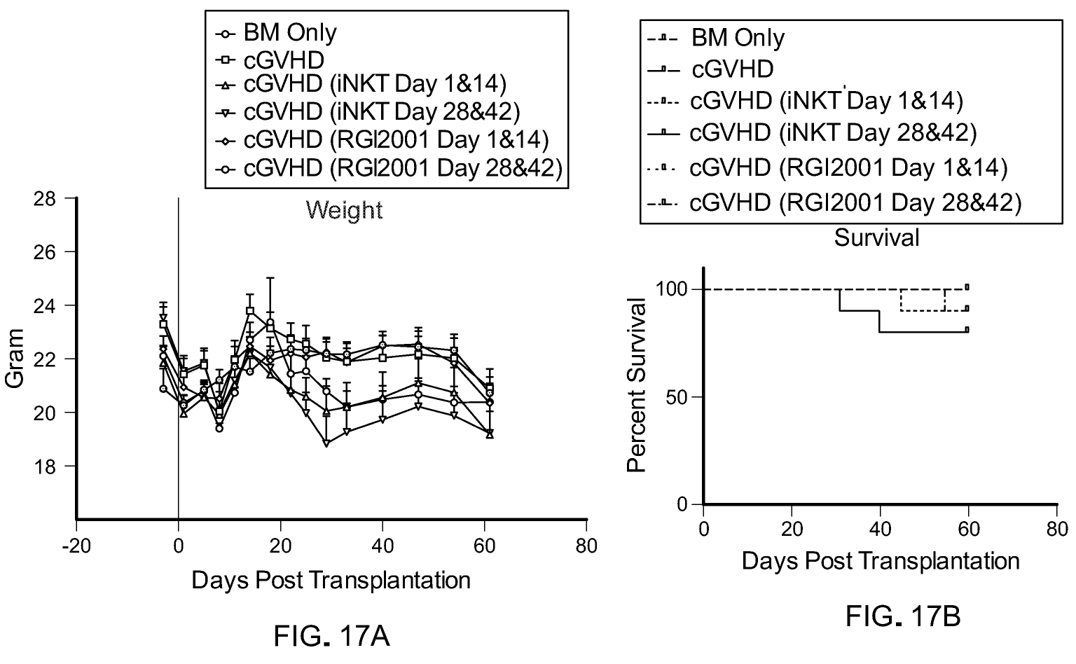

FIG. 17 are graphs showing the weight (Panel A) and survival (Panel B) of the mice. As can be seen by FIG. 17, the results of these experiments suggest that chronic GVHD, rather than acute GVHD, is more prevalent in mice, where more weight loss and death would occur.

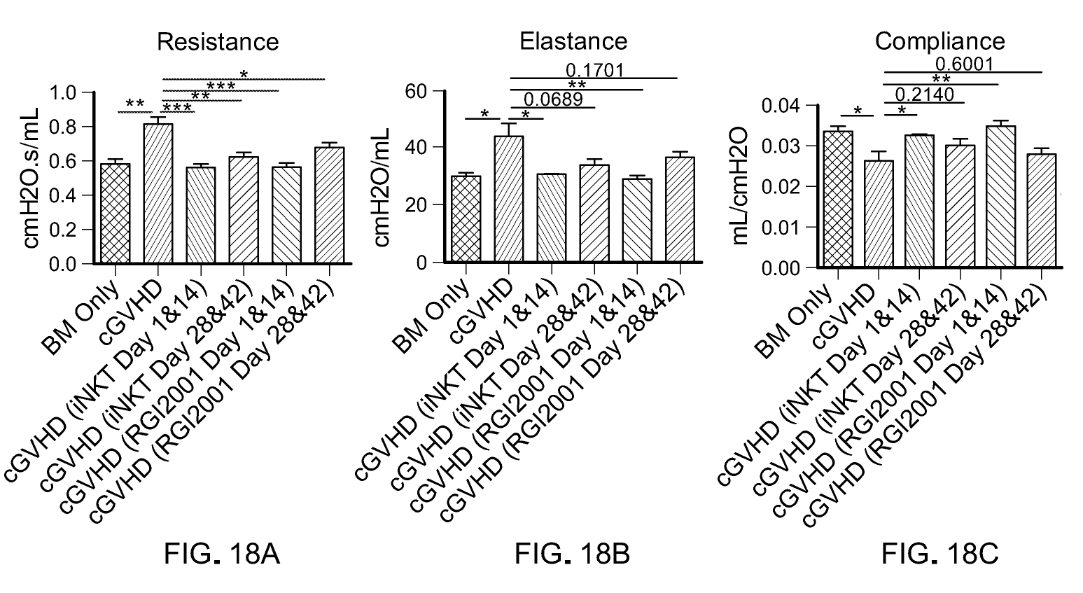

Five to eight mice from each group were analyzed for lung disease via pulmonary function, and these results are presented in FIG. 18. FIG. 18 demonstrates that both prophylactic and therapeutic iNKT infusion blocked cGVHD. The same effect was attained by activating endogenous iNKT cells using RGI2001.

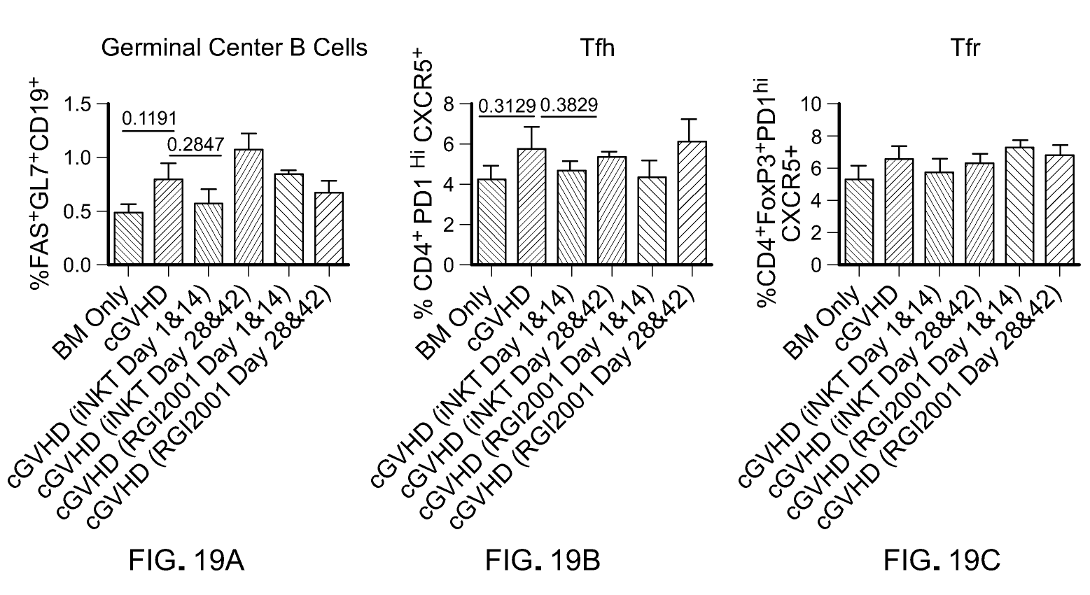

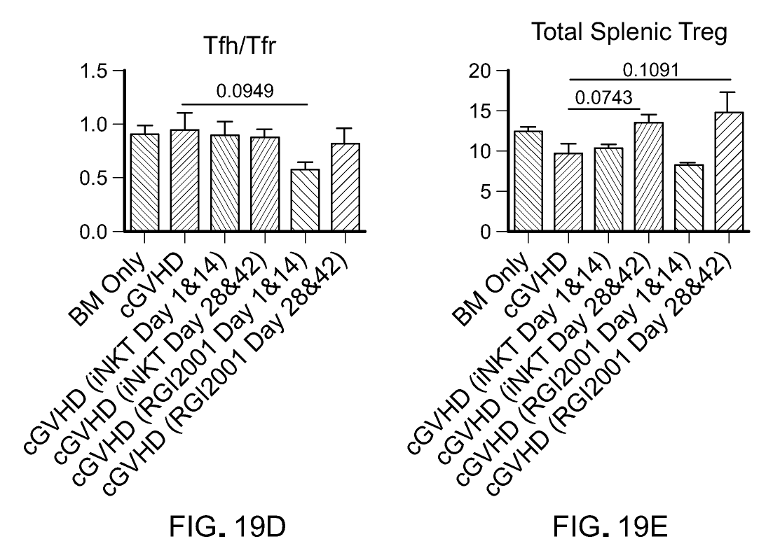

FIG. 19 are the results of flow cytometry analysis. The control groups in these experiments weren't as expected; it is possible that these mice didn't develop a “typical” germinal center-driven cGVHD, and iNKT cells and RGI2001 did not restore lung function, supporting the idea of a germinal center-independent pathway used by iNKT cells. HE and Trichrome staining are used to further evaluate these mice.

In summary, these experiments demonstrated that infusion with iNKT cells prevented or treated cGVHD to a similar extent than injection with RGI2001. For both iNKT cells and RGI2001, prophylaxis resulted a better improvement than treatment. Frozen lung, liver, spleen and colon tissues were saved for further analysis.

### Example 4

### Evaluation of the iNKT Population in cGVHD Mice

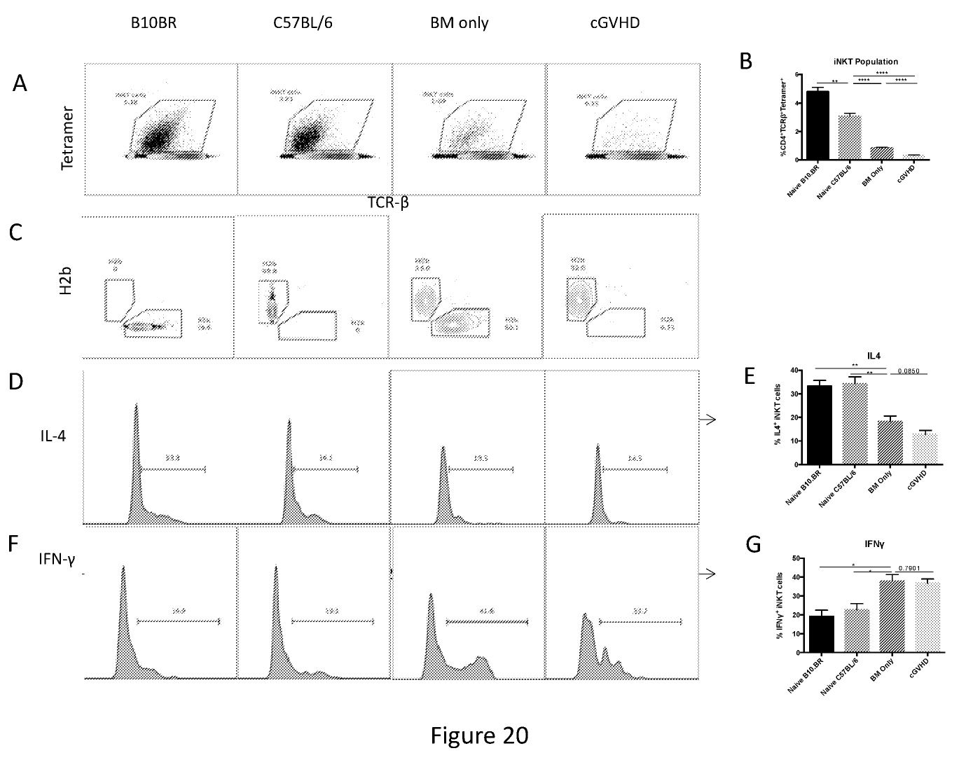

Experiments were performed to determine whether cGVHD mice, at day 28, have a defective iNKT population. See FIG. 20. Three B10BR mice, five B6 mice, five BM only mice, and five cGVHD mice were analyzed for their iNKT population (Panel B). Splenocytes were harvested and stained with anti-CD4, anti-TCR-β, CD1d-Tetramer (Panel A) and fixable viability dye (Panel C). Flow analysis showed a decreased iNKT cell population in cGVHD mice compared with naïve mice and BM only mice (Panels D, E, F and G). Five million splenocytes were stimulated with PMA for 4 h in complete cell culture media. After stimulation, cells were stained with the same surface markers followed by fixation for 20 min. Cells were stained with anti-IL4 for 20 min. Flow showed a decreased IL4 production in cGVHD mice.

In summary, 28 days after transplantation, both BM and cGVHD groups had a significantly reduced percentage of iNKT cells than naïve mice, and the cGVHD group had significantly reduced iNKT cells than the BM only group (Panel B). In addition, iNKT cells from transplanted mice produced more IFN-gamma than naive mice, but there was no difference between BM only mice and cGVHD mice in IFN-gamma production (Panel F and G). On the other hand, iNKT cells from transplanted mice produced less IL-4 than naïve mice, and iNKT cells from cGVHD mice produced less IL-4 than that from BM mice (Panel D and E; p=0.085).

### Example 5

### Experiments to Determine Which Treg Compartment is Required to Reduce cGVHD

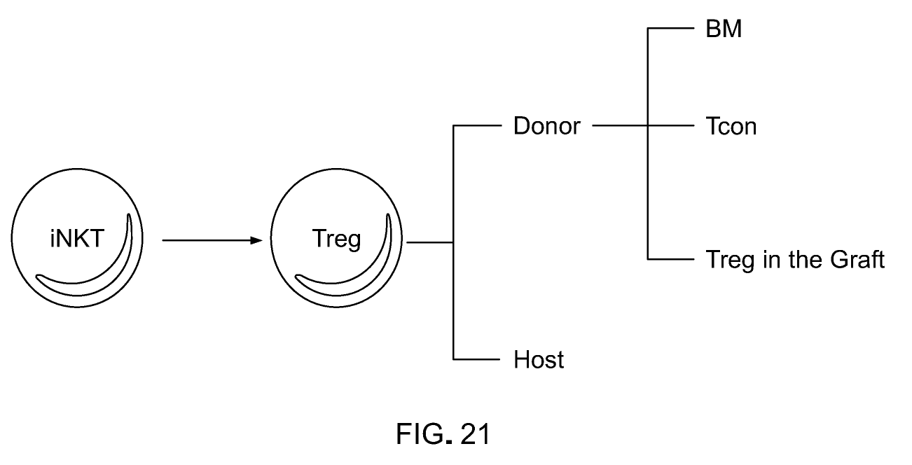

Experiments were performed to determine which Treg compartment is involved in reducing cGVHD. FIG. 21 shows the experimental plan.

The treatment regimen is shown below in Table 4. Briefly, 100K iNKT cells, obtained as described herein, were infused into the recipient mice on day 28 and 42 after bone marrow transplantation to and from the indicated strain. 0.1 mcg DT per mouse was injected on days −3, −1, 1 & 3 relative to iNKT infusion.

TABLE 4

| | | Donor BM | Donor T | | Cy | | T cell | |
|---|---|---|---|---|---|---|---|---|
| Group | N | 8 million | cells | Recipient | (d −3, −2) | X-Ray | Dose | Treatment |
| 1 | 8 | B6 Foxp3 | — | B10.BR | 120 mg/kg | 830 | — | — |
| | | Luci DTR4 | | | | | | |
| 2 | 12 | B6 Foxp3 | B6 Foxp3 | B10.BR | 120 mg/kg | 830 | 72500 | — |
| | | Luci DTR4 | Luci DTR4 | | | | | |
| 3 | 12 | B6 Foxp3 | B6 Foxp3 | B10.BR | 120 mg/kg | 830 | 72500 | DT injection |
| | | Luci DTR4 | Luci DTR4 | | | | | |
| 4 | 12 | B6 Foxp3 | B6 Foxp3 | B10.BR | 120 mg/kg | 830 | 72500 | 100K iNKT |
| | | Luci DTR4 | Luci DTR4 | | | | | Day 28 & 42 |
| 5 | 12 | B6 Foxp3 | B6 Foxp3 | B10.BR | 120 mg/kg | 830 | 72500 | 100K iNKT |
| | | Luci DTR4 | Luci DTR4 | | | | | Day 28 & 42 |
| | | | | | | | | DT injection |

Five mice from Group 2 and 4 underwent BLI (day 35 & 42). These mice also were used for PFT studies on day 56, provided they appeared healthy. Also on day 56, flow cytometry was performed to evaluate GC B cells, Tfr, Tfh and total T cells. Staining panels included donor BM, donor T cells, and host markers so as to examine each compartment. Lung, liver, spleen, and colon were collected for histology, trichrome staining and hydroxyproline assay.

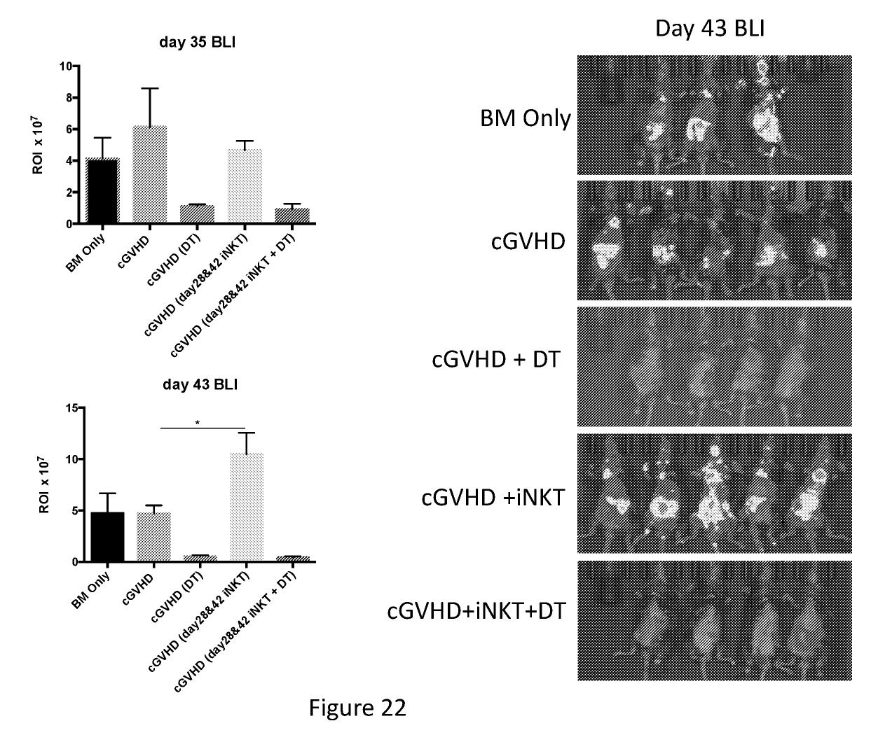

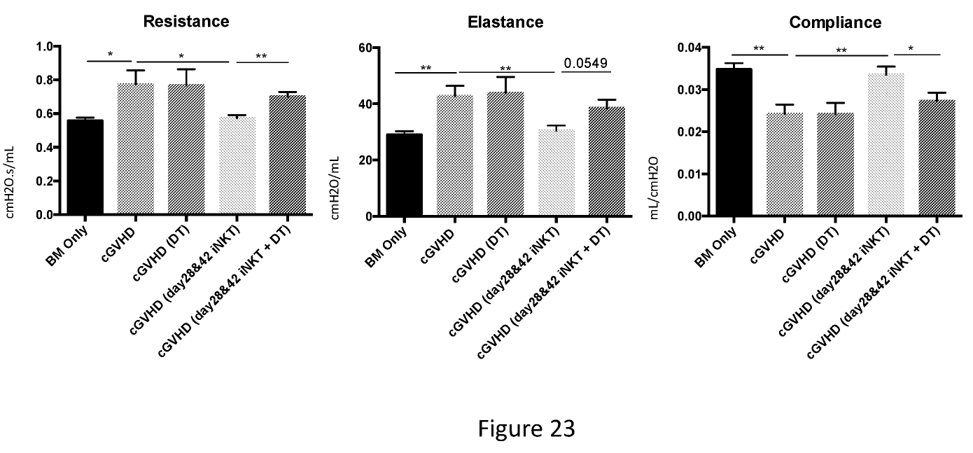

Infused iNKT cells disseminated in the recipients and were identified from recipients' lung, liver and spleen (FIG. 22). Mechanistically, iNKT cells controlled the spontaneous germinal center reactions that drive cGVHD pathogenesis by expanding donor Tregs and increasing follicular Treg density in the germinal center areas. See FIG. 23.

### Example 6

### Agonists of iNKT

Intravascular administration of alpha-galactosylceramide (alpha-Galcer), a potent agonist of iNKT cells, was able to prevent or reverse cGVHD.

These studies demonstrate the role of iNKT cells in regulating cGVHD pathogenesis and highlight the potential of both iNKT cells and alpha-Galcer as novel therapies for cGVHD.

### Example 7

### iNKT Cells Ameliorate Chronic GVHD in Mice by Expanding Donor Treg Cells

### Mice and Bone Marrow (BM) Transplantation

C57BL/6 (B6) (Charles River), B10.BR (JAX®), CXCR5−/− and IL4−/− on B6 background (JAX®) and B6.Foxp3.Luci.DTR-4 mice (gift from Professor Gunter Hammerling) were housed in a pathogen-free facility and used with IACUC approval. To induce cGVHD, B10.BR mice were given cyclophosphamide and total body irradiation pre-transplant and B6 bone marrow (BM) only (no cGVHD) or with 75,000 purified T-cells (cGVHD) (Flynn et al., 2014, Blood, 123:3988-98).

iNKT Isolation and Treg Depletion

iNKT cells were FACS-sorted from CD1d-PBS57 tetramer enriched B6 splenocytes (Schneidawind et al., 2014, Blood, 125(22):3491-500) to high purity (>95%) and maintained cytokine-producing function (not shown). iNKTs were infused at 50,000 or 100,000 doses at the indicated times. Where stated, Tregs were depleted in B6.Foxp3.Luc-DTR-4 mice by diphtheria toxin (DT) (0.1 microgram/mouse) injections before and after iNKTs infusion (days −2, −1, 1 & 2).

cGVHD Evaluation

cGVHD was evaluated by pulmonary functional tests (PFTs) and trichrome staining (Flynn et al., 2014, Blood, 123:3988-98; Srinivasan et al., 2012, Blood, 119(6):1570-80). Lung hydroxyproline quantification was per manufacturer's instructions (Sigma MAK008). GC reaction was evaluated by immunofluorescence staining and flow cytometry (Flynn et al., 2014, Blood, 123:3988-98). Bioluminescent imaging (BLI) was performed using IVIS Spectrum.

Therapeutic iNKT Cell Infusion Reversed Established cGVHD

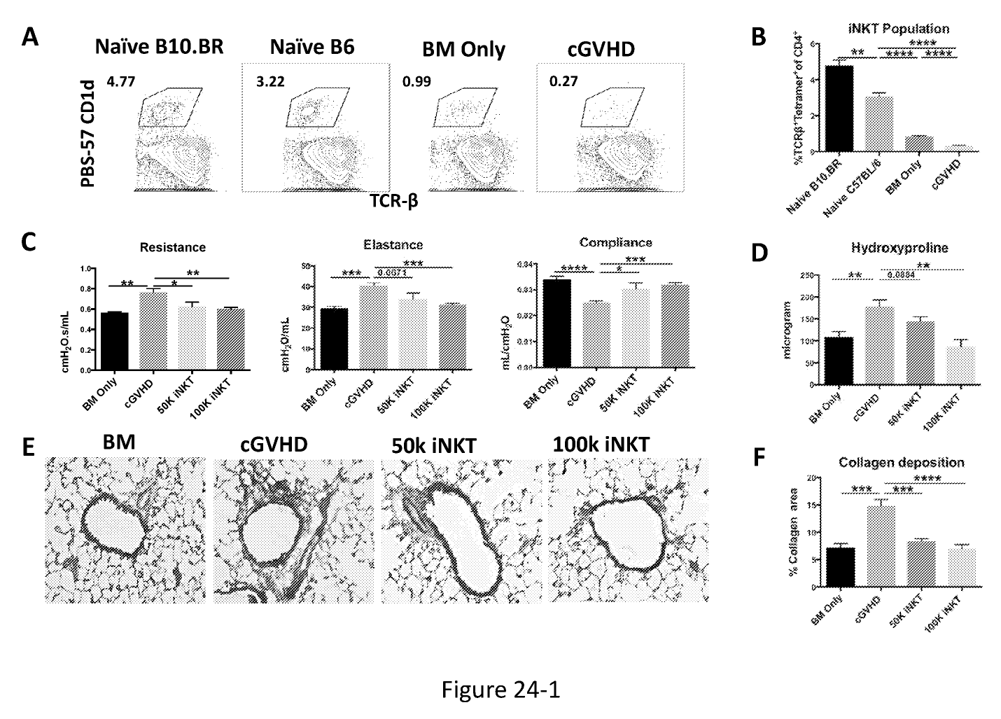

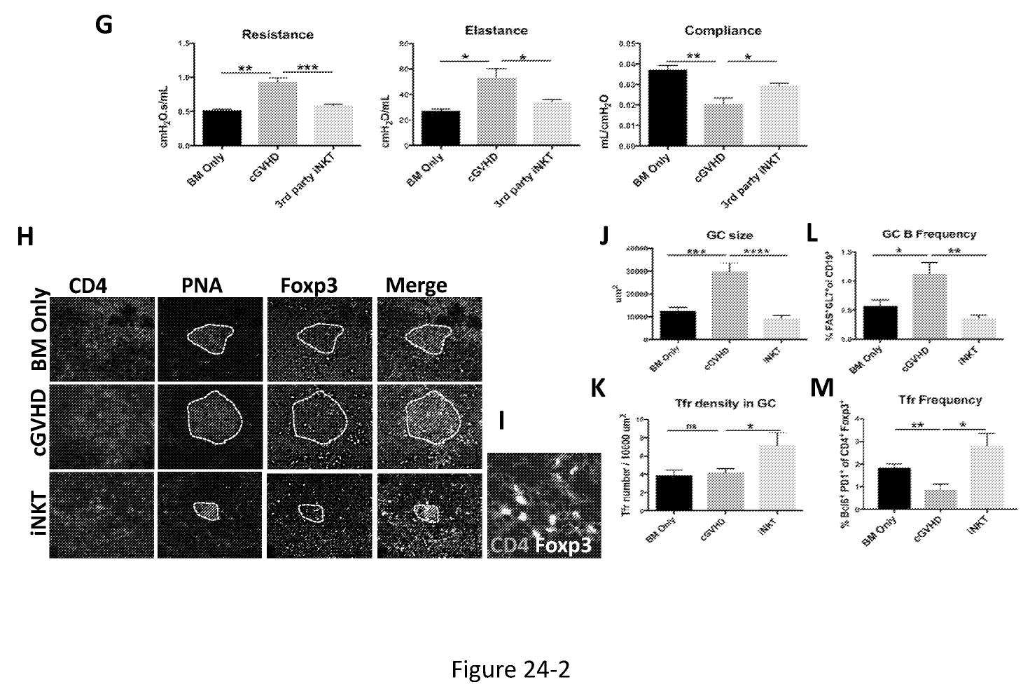

To examine whether the iNKT pool in cGVHD is deficient, splenic iNKTs were analyzed from early phase cGVHD mice (day 28) and naïve donor and host strain mice. cGVHD mice have a significantly lower splenic iNKTs than naïve mice and BM controls (FIG. 24A-24B). Thus, adoptive iNKT transfer was explored to treat cGVHD. Donor iNKTs were infused to cGVHD on day 28 & day 42 post-transplantation. iNKTs (50,000 or 100,000 cells) reversed lung cGVHD measured by PFTs (FIG. 24C). Total lung hydroxyproline (FIG. 24D), collagen deposition around peri-bronchial and perivascular areas (FIGS. 24E & 24F) were reduced (50,000 cells) or completely reversed (100,000 cells, this dose was used for subsequent studies). iNKT cells form a third strain (Balb/c) reversed cGVHD as well as iNKT from donor strain (FIG. 24G). iNKT infusion reduced GC area (FIGS. 24H & 24J) and increased Tfr density (FIGS. 24I & 24K). Flow cytometry analysis of day 55 splenocytes confirmed that iNKT reduced GC B and increased Tfr frequency (FIGS. 24L & 24M). Thus, donor iNKT reversal of established cGVHD was likely a result of increased Tfr frequency.

iNKT Reversed cGVHD through Donor Treg Expansion

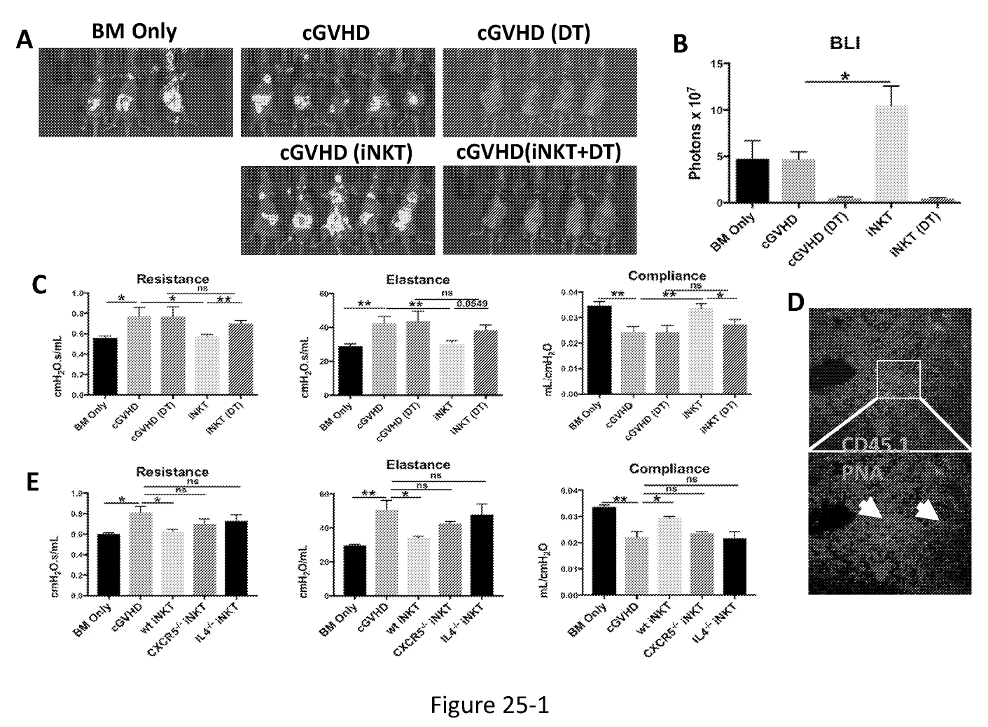

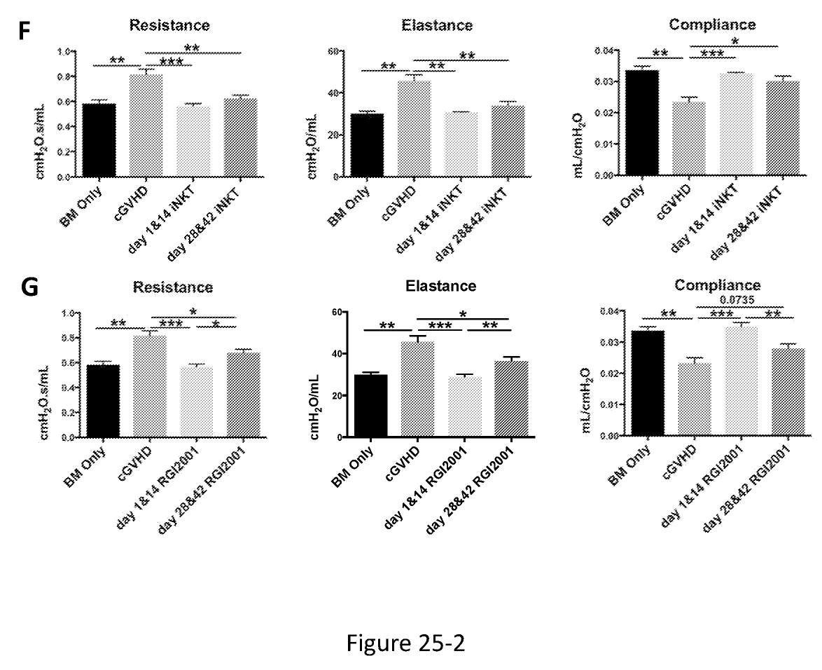

Due to the reduced Treg frequency observed in both cGVHD patients (Zorn et al., 2005, Transplantation, 106(8):2903-11) and a murine model (McDonald-Hyman et al., 2016, Blood, 128(7):1013-17), and reversal of cGVHD in murine model with Treg infusion (McDonald-Hyman et al., 2016, Blood, 128(7):1013-17), it was examined whether iNKT reversed cGVHD through Treg expansion. Donor B6.Foxp3.Luci.DTR-4 mice (Suffner et al., 2010, J. Immunol., 184(4):1810-20) permitted both tracking expansion and eliminating Foxp3 expressing Tregs; iNKT increased Foxp3 signal intensity by 2-fold (FIGS. 25A & 25B). Peri-infusion donor Treg depletion completely abrogated iNKT-mediated protection as indicated by PFTs (FIG. 25C), confirming a Treg dependent mechanism.

Infused CD45.1 iNKT cells were detected in GC areas 5 days after infusion (FIG. 25D). To test whether GC migration and IL4 production are required for iNKT's protective function in cGVHD, iNKTs from CXCRS−/− or IL4−/− mice were infused. Neither CXCRS−/− nor IL4−/− iNKTs were able to reverse cGVHD (FIG. 25E). Taken together with the high Tfr frequency conferred by iNKT infusion, these data point to in situ Tfr expansion in GC area by iNKT through IL4 dependent mechanism as a key mechanistic underpinning of iNKT-mediated cGVHD therapy.

Pharmacologic Activation of iNKT is Effective in Preventing and Reversing cGVHD

To determine the potential of iNKT in preventing cGVHD, donor iNKTs were given on day 1 & day 14 (prophylaxis) or on day 28 & day 42 (therapy). PFTs showed that prophylactic infusion completely blocked cGVHD, resulting in modestly more robust protection compared with therapeutic infusion (FIG. 25F). Prophylactic iNKT efficacy may be advantageous due to the expansion of host radio-resistant Tregs or donor Tregs in the graft that suppress inflammation and tissue damage, preventing cGVHD initiation, as well as easier disease prevention than reversal. In support of this latter hypothesis and further demonstrating the potential iNKT therapeutic benefits, the efficacy of RGI2001, a liposomal formulation of a-galactosylceramide, was demonstrated in both treating and, to an even greater extent, in preventing, cGVHD (FIG. 25G).

Because aGVHD is a critical risk factor for cGVHD1, managing aGVHD can significantly reduce cGVHD incidence. iNKT infusion (25,000-100,000 cells) protected mice from aGVHD in a dose-dependent manner through Treg expansion (Schneidawind et al., 2014, Blood, 124(22):3320-9). These data suggest that infusion of iNKT cells protects from both aGVHD and cGVHD, which likely is due to the fact that both diseases are associated with an inadequate Treg pool and, hence, T effector/Treg ratio. The fact that iNKT infusion is useful for both aGVHD and cGVHD offers the possibility for optimal treatment of patients with dual acute and chronic GVHD components.

Compared to Treg infusion, iNKT infusion required fewer cells (100,000 iNKT versus 500,000 Treg in the same model) to reach optimal effect (McDonald-Hyman et al., 2016, Blood, 128(7):1013-7; Guan et al., 2016, Bone Marrow Transplant., 1-9). Additionally, iNKT cells have inherent anti-viral and anti-tumor abilities (Brennan et al., 2013, Nat. Rev. Immunol., 13(2):101-17) that are desirable for cGVHD patients. iNKT cells are persistent in a host, as they can be detected in spleen, liver and lung at least 2 weeks after infusion (not shown). This study provides evidence that iNKT infusion and expansion are promising prophylactic and therapeutic options for cGVHD patients.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of reducing chronic graft-versus-host-disease (cGVHD) in a patient, wherein the patient is a recipient of a transplant from a donor, comprising:
identifying a patient suffering from cGVHD;
providing donor iNKT cells; and
administering the donor iNKT cells to the patient.

2. The method of claim 1, wherein the donor iNKT cells are administered to the patient one time.

3. The method of claim 1, wherein the donor iNKT cells are administered to the patient two times.

4. The method of claim 1, wherein the administering is by infusion.

5. The method of claim 1, wherein the donor iNKT cells are administered to the patient by infusion.

6. The method of claim 1, wherein the transplant is a bone marrow transplant, a hematopoietic stem cell transplant, or a progenitor cell transplant.

7. The method of claim 1, further comprising expanding the iNKT cells prior to the administering step.

8. The method of claim 1, further comprising contacting the donor iNKT cells with RGI-2001 prior to the administering step.

9. A method of treating an autoimmune disease or an alloimmune disease in a patient, comprising:
identifying a patient suffering from an autoimmune disease or an alloimmune disease;
providing donor iNKT cells; and
administering at least one dose of donor iNKT cells to the patient.

10. The method of claim 9, wherein the autoimmune disease or the alloimmune disease is selected from the group consisting of lupus, arthritic, immune complex glomerulonephritis, goodpasture, uveitis, and multiple sclerosis.

11. The method of claim 9, wherein the donor iNKT cells are administered to the patient one time.

12. The method of claim 9, wherein the donor iNKT cells are administered to the patient two times.

13. The method of claim 9, wherein the administering is by infusion.

14. The method of claim 9, wherein the donor iNKT cells are administered to the patient by infusion.

15. The method of claim 9, wherein the transplant is a bone marrow transplant, a hematopoietic stem cell transplant, or a progenitor cell transplant.

16. The method of claim 9, further comprising expanding the donor iNKT cells prior to the administering step.

17. The method of claim 9, further comprising contacting the iNKT cells with RGI-2001 prior to the administering step.

18. A method of reducing chronic graft-versus-host-disease (cGVHD) in a patient, comprising:
administering a therapeutic amount of an agonist of iNKT cells to the patient.
